(19) United States
(12) Patent Application Publication
(10) Pub. No.: US 2023/0031235 A1
(43) Pub. Date: Feb. 2, 2023

(54) PROCESS FOR THE COSMETIC TREATMENT OF KERATIN FIBRES USING A RARE-EARTH METAL AND A PARTICULAR POLYMER

(71) Applicant: L'OREAL, Paris (FR)

(72) Inventors: Valérie JEANNE-ROSE, AULNAY-SOUS-BOIS (FR); Laurent SABATIE, AULNAY-SOUS-BOIS (FR); Henri SAMAIN, CHEVILLY-LARUE (FR)

(73) Assignee: L'OREAL, Paris (FR)

(21) Appl. No.: 17/787,192

(22) PCT Filed: Dec. 18, 2020
(86) PCT No.: PCT/EP2020/087274

§ 371 Date: Jun. 17, 2022

(30) Foreign Application Priority Data
Dec. 20, 2019 (FR) 1915294

(51) Int. Cl. A61K 8/19; A61K 8/81; A61Q 5/00

(52) U.S. Cl. CPC A61K 8/19; A61K 8/8141; A61Q 5/002

(57) ABSTRACT

The invention relates to a cosmetic process for treating keratin fibres, in particular human keratin fibres such as the hair, using at least one particular metal compound belonging to the group of the rare-earth metals and at least one fixing polymer.

The invention relates to a cosmetic process for treating keratin fibres, in particular human keratin fibres such as the hair, using at least one particular metal compound and at least one fixing polymer.

The present invention also relates to a multi-compartment kit containing said particular metal compound and said fixing polymer.

Many people use hairstyling processes in order to construct and/or structure their hairstyle and to give it hold. To do this, they usually use products that are usually in the form of lotions, gels, mousses, creams, sprays, etc. These compositions generally comprise one or more film-forming polymers. These polymers allow the formation of a coating film on the hair, and/or the formation of micro-welds between the individual hairs, thus ensuring the hairstyle hold.

These compositions are subjected to several stress factors throughout the day, for example heat, humidity, rain or sebum, and repeated mild mechanical stress factors, which may bring about a loss of fixing of the hairstyle after only a few hours.

Moreover, the processes used are not always entirely satisfactory in terms of hairstyle hold. It has notably been found that the hairstyle collapses after 3 to 4 hours of exposure to stress factors.

Furthermore, the compositions used in these processes also have a certain number of drawbacks. These compositions generally lead to a dry feel, a dull and coarse appearance of the hair, and have a tendency to solidify the hairstyle, notably giving a “helmet effect”, which is often poorly perceived by users.

There is thus a real need to develop cosmetic processes for treating keratin fibres, in particular human keratin fibres such as the hair, which can give manageability and also strong and long-lasting fixing to the head of hair. These processes must also impart good styling effects, such as giving the keratin fibres body and volume, which are capable of persisting throughout the day, while at the same time allowing the hairstyle to maintain a natural, non-solidified appearance. These processes must also afford satisfactory cosmetic properties, in particular a pleasant cosmetic feel, notably a soft, smooth feel.

It has been discovered, surprisingly, that a cosmetic process for treating keratin fibres, in particular human keratin fibres such as the hair, comprising the application of a composition comprising at least one compound of a metal belonging to the group of the rare-earth metals, and the application of a composition comprising at least one fixing polymer, makes it possible to achieve the above objectives, notably in terms of manageability, fixing, styling effect and cosmetic properties.

One subject of the present invention is thus a cosmetic process for treating, in particular for styling and/or shaping, keratin materials, in particular human keratin materials such as the skin or the hair, comprising:
application to said keratin fibres of a composition (A) comprising one or more salts and/or complexes of a metal belonging to the group of the rare-earth metals in oxidation state +III; and
application to said keratin fibres of a composition (B) comprising one or more fixing polymers,

it being understood that composition (A) and composition (B) are applied to said keratin fibres together or sequentially.

In one embodiment, compositions (A) and (B) are applied together, i.e. “one-step process” or all-in-one process”, the compositions possibly being mixed beforehand (i.e. one-step process without mixing at the time of use) or at the time of use.

In another embodiment, compositions (A) and (B) are applied sequentially, i.e. “two-step process” or “sequential process”. In this embodiment, composition (A) may be applied before or after composition (B).

The process according to the invention, combining a compound of a particular metal with a fixing polymer, affords a substantial, long-lasting and immediate manageability and fixing effect, with additional styling effects, such as body and volume, to keratin fibres, which is capable of persisting over time and which is resistant to washing, notably to shampooing several times. The keratin fibres treated via the process of the invention also have satisfactory cosmetic properties when the product is applied regularly. Specifically, one advantage of the invention is that it makes it possible to obtain a substantial styling effect from the very first application, due to the coating it produces on the fibres, and then, in the course of the shampoo washes and reapplications, the body effect is reinforced to the point that the applications of the product can be spaced out or a lighter styling product can be applied thus tending towards a styling effect with little or no perception of coating.

A subject of the invention is also a multi-compartment kit or device comprising a first compartment containing a composition (A) comprising one or more salts and/or complexes of a metal belonging to the group of the rare-earth metals in oxidation state +III; and a second compartment containing a composition (B) comprising one or more fixing polymers. This kit or device then makes it possible to vary the amount of polymer in the product applied (if performed in one step) or the relative amounts of B relative to A applied (if performed sequentially).

Other subjects, characteristics, aspects and advantages of the invention will emerge even more clearly on reading the description and the examples that follow.

In the text hereinbelow, unless otherwise indicated, the limits of a range of values are included in that range, notably in the expressions “between” and “ranging from . . . to . . . ”.

Moreover, the expression “at least one” used in the present description is equivalent to the expression “one or more”.

Composition (A)

The process according to the present invention comprises the application to keratin fibres, in particular to human keratin fibres such as the hair, of a composition (A) comprising one or more salts and/or complexes of a metal belonging to the group of the rare-earth metals in oxidation state +III.

As examples of metals belonging to the group of the rare-earth metals M, mention may be made of scandium, yttrium, lanthanum, cerium, praseodymium, neodymium, promethium, samarium, europium, gadolinium, terbium, dysprosium, holmium, erbium, thulium, ytterbium and lutetium.

Preferably, the metal(s) belonging to the group of the rare-earth metals are chosen from cerium, yttrium, ytterbium, lanthanum and europium.

The rare-earth metal salts according to the invention may be soluble or insoluble in the composition containing them.

In the case of the salts and complexes of rare-earth metal according to the invention, the compound of the metal belonging to the group of the rare-earth metals is in oxidation state +III.

The metal M may then be associated, via its electron shell, to n1 anionic groups forming an ionic bond with M and/or to n2 groups forming a coordination bond with M. The groups forming a coordination bond are groups bearing a donor pair, for instance carbonyl or amine.

If n2=0, the compound of the metal belonging to the group of the rare-earth metals forms a salt and, in this case, the metal M is associated with three anionic groups.

If n2>0, the compound of the metal belonging to the group of the rare-earth metals forms a complex and, in this case, the number of anionic groups n1 may range from 0 to 3.

The metal M is associated with one or more anionic groups and/or one or more groups forming a coordination bond.

In the text hereinbelow, the term “ligand” refers to an ion or a molecule bearing a group which combines, via an ionic bond or a coordination bond, to the metal M. The same ligand may bear several groups.

The term “rare-earth metal compound” refers to the combination of the metal M with its ligand(s).

A definition of rare-earth metal salts or complexes may be found in: Progress in the Science and Technology of the Rare Earths, Part 1, published by Leroy Eyring in 1964, edited by Macmillan Company and written by F. Gaume-Mahn, page 259 et seq.

Ligands Associated with the Rare-Earth Metals M to Form a Salt or a Complex and Corresponding Rare-Earth Metal Compounds

a) Typically, the ligand may be a monoanionic ion, which may or may not be monoatomic, such as a nitrate, or a hydroxyl (OH⁻) or a halide (typically chloride or bromide). By way of example, the rare-earth metal compound resulting therefrom may then be MCl₃, M(OH)₃, M(NO₃)₃ etc. and in particular CeNO₃, YNO₃, LaNO₃, CeCl₃, YCl₃ or LaCl₃.

b) The ligand may be a dianionic or trianionic ion, such as phosphate or sulfate. By way of example, mention may be made of rare-earth metal compounds such as MPO₄, or M₂(SO₄)₃ and in particular CePO₄, YPO₄, LaPO₄, Ce₂(SO₄)₃, Y₂(SO₄)₃ or La₂(SO₄)₃.

c) The ligand may contain one or more groups forming a coordination bond and a function forming an ionic bond.

Thus, the ligand may be a monocarboxylate or polycarboxylate molecule, such as acetate or succinate. In this case, it is considered that the carboxylate function acts as an anionic group, by means of the hydroxyl of the carboxylic group, and acts as a group forming a coordination bond by means of the lone pair of the oxygen of the carbonyl function. Thus, the resulting rare-earth metal compound may be M(R—(COO)ₙ)_{3/n}. In addition to bearing one or more carboxylates, the ligand may include other functions, such as hydroxyls or amines. Thus, the ligand may be in the form of hydroxycarboxylic acids or aminocarboxylic acids. As monocarboxylic or polycarboxylic compound bearing additional functions, mention may be made of tartrate, citrate, glycolate or ethylenediaminetetraacetate (EDTA) ions.

The ligand may bear a non-localized anionic charge, for instance acetylacetonate. The rare-earth metal compound will then be M(acetylacetonate)₃ or M(acetylacetonate)₃.7H₂O in which each acetonate bonds to the metal M via its two carbonyl functions, one acting as an anionic group, the other as a group bonding by coordination.

It may also be of the aromatic type, such as a phenol, a cyclopentadiene (Progress in the Science and Technology of the Rare Earths, published by Leroy Eyring and written by F. Gaume-Mahn, page 296), or a pyridine.

The rare-earth metal compound may include one or more ligands forming a coordination bond and one or more ligands forming an ionic bond. Thus, the rare-earth metal compound may be yttrium dihydroxyacetate Y((OH)₂acetate) (Synthesis and Properties of Yttrium Hydroxyacetate Sols by S. S. Balabanov, E. M. Gavrishchuk, and D. A. Permin, published in the review Inorganic Materials, 2012, Vol. 48, No. 5, pages 500-503, in 2012).

The rare-earth metal compound may be a double salt, for example with a rare-earth metal M and another cation other than rare-earth metals, for instance an alkali metal (Li,Ce(SO₄)₂) or an alkaline-earth metal or a cationic organic species, such as a quaternary amine, such as an alkylpyridinium group.

These rare-earth metal compounds, which are often highly hygroscopic, are in the form of hydrates, for instance CeCl₃.7H₂O; YCl₃.6H₂O; LaCl₃.7H₂O; Ce(acetonate)₃.xH₂O.

Preferably, the salts and/or complexes of metals belonging to the group of the rare-earth metals in oxidation state +III are chosen from cerium chloride, yttrium chloride, lanthanum chloride, ytterbium chloride, europium chloride, and mixtures thereof.

Preferably, the compound of the metal belonging to the group of the rare-earth metals is dissolved in composition (A).

Advantageously, the compound(s) of the metal belonging to the group of the rare-earth metals are present in a total content ranging from 0.05% to 25% by weight, preferably from 0.2% to 15% by weight, relative to the total weight of composition (A).

Composition (A) is preferably aqueous, alcoholic or aqueous-alcoholic.

When composition (A) is aqueous, it preferably comprises water, in a content of greater than or equal to 50% by weight, more preferentially greater than or equal to 70% by weight, and even more preferentially greater than or equal to 90% by weight relative to the total weight of composition (A).

When composition (A) is alcoholic or aqueous-alcoholic, it may notably comprise one or more organic solvents, preferably in a content ranging from 0.05% to 95% by weight and even more preferentially from 1% to 70% by weight, relative to the total weight of composition (A).

This organic solvent may be a C₂ to C₄ lower alcohol, in particular ethanol or isopropanol, polyols and polyol ethers such as propylene glycol, polyethylene glycol or glycerol. The organic solvent is preferably ethanol.

Composition (A) may also comprise one or more surfactants chosen from cationic, anionic, nonionic, amphoteric or zwitterionic surfactants, and mixtures thereof.

Cationic Surfactants

The term “cationic surfactant” means a surfactant that is positively charged when it is contained in the compositions according to the invention. This surfactant may bear one or more positive permanent charges or may contain one or more cationizable functions within the compositions according to the invention.

The cationic surfactants are advantageously chosen from optionally polyoxyalkylenated primary, secondary or tertiary fatty amine salts, quaternary ammonium salts, and mixtures thereof.

As quaternary ammonium salts, mention may be made notably of:
the quaternary ammonium salts of formula (Ia):

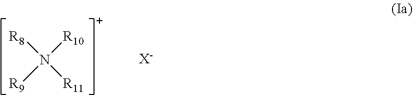

in which:

the groups R₈ to R₁₁, which may be identical or different, represent a linear or branched aliphatic group including from 1 to 30 carbon atoms, or an aromatic group such as aryl or alkylaryl, at least one of the groups R₈ to R₁₁ including from 8 to 30 and preferably from 12 to 24 carbon atoms, it being possible for the aliphatic groups to include heteroatoms notably such as oxygen, nitrogen, sulfur and halogens; and

X⁻ is an anion notably chosen from the group of halides, phosphates, acetates, lactates, (C₁-C₄)alkyl sulfates, (C₁-C₄)alkylsulfonates or (C₁-C₄)alkylarylsulfonates.

The aliphatic groups R₈ to R₁₁ may be chosen from C₁-C₃₀ alkyl, C₁-C₃₀ alkoxy, (C₂-C₆) polyoxyalkylene, C₁-C₃₀ alkylamide, (C₁₂-C₂₂)alkylamido(C₂-C₆)alkyl, (C₁₂-C₂₂)alkyl acetate, and C₁-C₃₀ hydroxyalkyl groups.

Mention may notably be made of tetraalkylammonium halides, notably chlorides, such as dialkyldimethylammonium or alkyltrimethylammonium chlorides in which the alkyl group includes from 12 to 22 carbon atoms, in particular behenyltrimethylammonium chloride, distearyldimethylammonium chloride, cetyltrimethylammonium chloride and benzyldimethylstearylammonium chloride.

Mention may also be made of palmitylamidopropyltrimethylammonium or stearamidopropyldimethyl-(myristyl acetate)-ammonium halides, and notably chlorides; notably the product sold under the name Ceraphyl® 70 by the company Van Dyk.
the quaternary ammonium salts of imidazoline of formula (IIa):

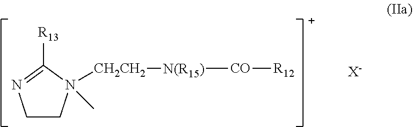

in which

R₁₂ represents an alkenyl or alkyl group including from 8 to 30 carbon atoms, for example derived from tallow fatty acids,

R₁₃ represents a hydrogen atom, a C₁-C₄ alkyl group or an alkenyl or alkyl group including from 8 to 30 carbon atoms,

R₁₄ represents a C₁-C₄ alkyl group,

R₁₅ represents a hydrogen atom or a C₁-C₄ alkyl group,

X⁻is an anion notably chosen from the group of halides, phosphates, acetates, lactates, (C₁-C₄)alkyl sulfates, (C₁-C₄)alkylsulfonates or (C₁-C₄)alkylarylsulfonates.

Preferably, R₁₂ and R₁₃ denote a mixture of alkenyl or alkyl groups including from 12 to 21 carbon atoms, for example derived from tallow fatty acids, R₁₄ denotes a methyl group and R₁₅ denotes a hydrogen atom. Such a product is sold, for example, under the name Rewoquat® W75 or W90 by the company Evonik.
the quaternary di- or triammonium salts of formula (IIIa):

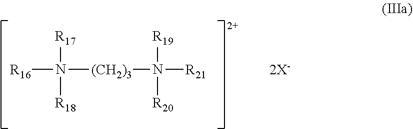

in which:
R₁₆ denotes an alkyl group including from 16 to 30 carbon atoms, which is optionally hydroxylated and/or optionally interrupted with one or more oxygen atoms,
R₁₇ denotes hydrogen, an alkyl group including from 1 to 4 carbon atoms or a group —(CH₂)₃—N⁺(R₁₆ₐ)(R₁₇ₐ)(R₁₈ₐ); R₁₆ₐ, R₁₇ₐ and R₁₈ₐ, which may be identical or different, denoting hydrogen or an alkyl group including from 1 to 4 carbon atoms,
R₁₈, R₁₉, R₂₀ and R₂₁, which may be identical or different, denote hydrogen or an alkyl group including from 1 to 4 carbon atoms, and
X⁻is an anion, chosen notably from the group of halides, acetates, phosphates, nitrates, (C₁-C₄)alkyl sulfates, (C₁-C₄)alkylsulfonates and (C₁-C₄)alkylarylsulfonates, in particular methyl sulfate and ethyl sulfate.

Such compounds are, for example, Finquat CT-P (Quaternium 89) and Finquat CT (Quaternium 75), sold by the company Finetex.
quaternary ammonium salts containing one or more ester functions, of formula (IVa) below:

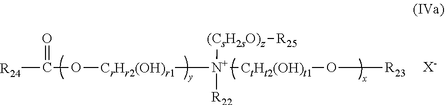

in which:
R₂₂ is chosen from C₁-C₆ alkyl groups and C₁-C₆ hydroxyalkyl or dihydroxyalkyl groups,
R₂₃ is chosen from the group R₂₆—C(═O)—; linear or branched, saturated or unsaturated C₁-C₂₂ hydrocarbon-based groups R₂₇; and a hydrogen atom,
R₂₅ is chosen from the group R₂₈—C(═O)—; linear or branched, saturated or unsaturated C₁-C₆ hydrocarbon-based groups R₂₉; and a hydrogen atom,
R₂₄, R₂₆ and R₂₈, which may be identical or different, are chosen from linear or branched, saturated or unsaturated C₇-C₂₁ hydrocarbon-based groups,
r, s and t, which may be identical or different, are integers ranging from 2 to 6,
r1 and t1, which may be identical or different, are equal to 0 or 1,
y is an integer ranging from 1 to 10,
x and z, which may be identical or different, are integers ranging from 0 to 10,
X⁻is an anion,

it being understood that r2+r1=2r and t1+t2=2t, and that the sum x+y+z ranges from 1 to 15,

with the proviso that when x=0 then R₂₃ denotes R₂₇ and that when z=0 then R₂₅ denotes R₂₉.

The alkyl groups R₂₂ may be linear or branched, preferably linear. Preferably, R₂₂ denotes a methyl, ethyl, hydroxyethyl or dihydroxypropyl group, and more particularly a methyl or ethyl group.

Advantageously, the sum x+y+z is from 1 to 10.

When R₂₃ is a hydrocarbon-based group R₂₇, it may comprise from 12 to 22 carbon atoms, or else may comprise from 1 to 3 carbon atoms.

When R₂₅ is a hydrocarbon-based group R₂₉, it preferably contains 1 to 3 carbon atoms.

Advantageously, R₂₄, R₂₆ and R₂₈, which may be identical or different, are chosen from linear or branched, saturated or unsaturated C₁₁-C₂₁ hydrocarbon-based groups, and more particularly from linear or branched C₁₁-C₂₁ alkyl and alkenyl groups.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻is preferably a halide, preferably chloride, bromide or iodide, a (C₁-C₄)alkyl sulfate, a (C₁-C₄)alkylsulfonate or a (C₁-C₄)alkylarylsulfonate, a methanesulfonate, a phosphate, a nitrate, a tosylate, an anion derived from organic acid such as an acetate or a lactate or any other anion that is compatible with the ammonium bearing an ester function. The anion X⁻is more particularly a chloride, a methyl sulfate or an ethyl sulfate.

Use is more particularly made, in the composition according to the invention, of the ammonium salts of formula (IVa) in which:
R₂₂ denotes a methyl or ethyl group,
x and y are equal to 1,
z is equal to 0 or 1,
r, s and t are equal to 2,
R₂₃ is chosen from the group R₂₆—C(═O)—; methyl, ethyl or C₁₄-C₂₂ hydrocarbon-based groups, and a hydrogen atom,
R₂₅ is chosen from the group R₂₈—C(═O)—; and a hydrogen atom,
R₂₄, R₂₆ and R₂₈, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups.

Advantageously, the hydrocarbon-based groups are linear.

Among the compounds of formula (IVa), mention may be made of the salts, notably the chloride or methyl sulfate of diacyloxyethyldimethylammonium, diacyloxyethylhydroxyethylmethylammonium, monoacyloxyethyldihydroxyethylmethylammonium, triacyloxyethylmethylammonium or mono acyloxyethylhydroxyethyldimethylammonium, and mixtures thereof. The acyl groups preferably contain 14 to 18 carbon atoms and are derived more particularly from a plant oil such as palm oil or sunflower oil. When the compound contains several acyl groups, these groups may be identical or different.

These products are obtained, for example, by direct esterification of triethanolamine, triisopropanolamine, alkyldiethanolamine or alkyldiisopropanolamine, which are optionally oxyalkylenated, with fatty acids or with fatty acid mixtures notably of plant or animal origin, or by transesterification of the methyl esters thereof. This esterification may be followed by a quaternization by means of an alkylating agent such as an alkyl halide, preferably methyl or ethyl halide, a dialkyl sulfate, preferably dimethyl or diethyl sulfate, methyl methanesulfonate, methyl para-toluenesulfonate, glycol chlorohydrin or glycerol chlorohydrin. Such compounds are sold, for example, under the names Dehyquart® by the company Henkel, Stepanquat® by the company Stepan, Noxamium® by the company CECA or Rewoquat® WE 18 by the company Evonik.

The composition according to the invention may contain, for example, a mixture of quaternary ammonium monoester, diester and triester salts with a weight majority of diester salts. Use may also be made of the ammonium salts containing at least one ester function that are described in U.S. Pat. Nos. 4,874,554 and 4,137,180. Use may also be made of the behenoylhydroxypropyltrimethylammonium chloride sold, for example, by the company Kao under the name Quartamin BTC 131.

Preferably, the ammonium salts containing at least one ester function contain two ester functions.

The cationic surfactant(s) are advantageously chosen from cetyltrimethylammonium, behenyltrimethylammonium and dipalmitoylethylhydroxyethylmethylammonium salts and mixtures thereof; more preferentially from behenyltrimethylammonium chloride or methosulfate, cetyltrimethylammonium chloride or methosulfate, dipalmitoylethylhydroxyethylmethylammonium chloride or methosulfate, and mixtures thereof.

Anionic Surfactants

The term “anionic surfactant” means a surfactant including, as ionic or ionizable groups, only anionic groups.

In the present description, a species is termed as being “anionic” when it bears at least one permanent negative charge or when it can be ionized as a negatively charged species, under the conditions of use of the composition of the invention (for example the medium or the pH) and not comprising any cationic charge.

The anionic surfactants may be chosen from sulfate, sulfonate and/or carboxylic (or carboxylate) surfactants. Needless to say, a mixture of these surfactants may be used.

It is understood in the present description that:
the carboxylate anionic surfactants comprise at least one carboxylic or carboxylate function (—COOH or —COO⁻) and may optionally also comprise one or more sulfate and/or sulfonate functions;
the sulfonate anionic surfactants comprise at least one sulfonate function (—SO₃H or —SO₃⁻) and may optionally also comprise one or more sulfate functions, but do not comprise any carboxylate functions; and
the sulfate anionic surfactants comprise at least one sulfate function but do not comprise any carboxylate or sulfonate functions.

The carboxylate anionic surfactants that may be used thus include at least one carboxylic or carboxylate function (—COOH or —COO⁻).

They may be chosen from the following compounds: fatty acids, acylglycinates, acyllactylates, acylsarcosinates, acylglutamates; alkyl-D-galactosideuronic acids, alkyl ether carboxylic acids, alkyl(C₆-C₃₀ aryl) ether carboxylic acids, alkylamido ether carboxylic acids; and also the salts of these compounds; and mixtures thereof;

the alkyl and/or acyl groups of these compounds including from 6 to 30 carbon atoms, notably from 12 to 28, even better still from 14 to 24 or even from 16 to 22 carbon atoms; the aryl group preferably denoting a phenyl or benzyl group;

these compounds possibly being polyoxyalkylenated, notably polyoxyethylenated, and then preferably including from 1 to 50 ethylene oxide units and better still from 2 to 10 ethylene oxide units.

Use may also be made of C₆-C₂₄ alkyl monoesters of polyglycoside-polycarboxylic acids such as C₆-C₂₄ alkyl polyglycoside-citrates, C₆-C₂₄ alkyl polyglycoside-tartrates and C₆-C₂₄ alkyl polyglycoside-sulfosuccinates, and salts thereof.

Preferentially, the carboxylate anionic surfactants are chosen, alone or as a mixture, from:
fatty acids;
acylglutamates, notably of C₆-C₂₄ or even C₁₂-C₂₀, such as stearoylglutamates, and in particular disodium stearoylglutamate;
acylsarcosinates, notably of C₆-C₂₄ or even C₁₂-C₂₀, such as palmitoylsarcosinates, and in particular sodium palmitoylsarcosinate;
acyllactylates, notably of C₁₂-C₂₈ or even C₁₄-C₂₄, such as behenoyllactylates, and in particular sodium behenoyllactylate;
C₆-C₂₄ and notably C₁₂-C₂₀ acylglycinates;
(C₆-C₂₄)alkyl ether carboxylates, and notably (C₁₂-C₂₀)alkyl ether carboxylates;
polyoxyalkylenated (C₆-C₂₄)alkyl(amido) ether carboxylic acids, in particular those including from 2 to 50 ethylene oxide groups;

in particular in the form of alkali metal or alkaline-earth metal, ammonium or amino alcohol salts.

Among the above carboxylic surfactants, mention may be made most particularly of surfactants of fatty acid type, notably of C₆-C₃₀. These surfactants are preferably chosen from the compounds of formula (a) below: R—C(O)—OX (a)

with
X denoting a hydrogen atom, an ammonium ion, an ion derived from an alkali metal or an alkaline-earth metal or an ion derived from an organic amine, preferably a hydrogen atom, and
R denoting a linear or branched, saturated or unsaturated alkyl group of 7 to 29 carbon atoms.

Preferably, R denotes a linear or branched, saturated or unsaturated alkyl group of 7 to 23 carbon atoms, preferably of 11 to 21 carbon atoms.

Among the fatty acids, mention may be made of lauric acid, palmitic acid, myristic acid, stearic acid, oleic acid and behenic acid.

The fatty acids are advantageously chosen from palmitic acid, myristic acid, stearic acid, and mixtures thereof.

Among the above carboxylic surfactants, mention may be made most particularly of surfactants of sarcosinate type, notably chosen from (C₆-C₃₀)acyl sarcosinates of formula (I) below:

R—C(O)—N(CH₃)—CH₂—C(O)—OX (I)

with
X denoting a hydrogen atom, an ammonium ion, an ion derived from an alkali metal or an alkaline-earth metal or an ion derived from an organic amine, preferably a hydrogen atom, and
R denoting a linear or branched alkyl group of 6 to 30 carbon atoms.

Preferably, R denotes a linear or branched alkyl group of 8 to 24 carbon atoms, preferably of 12 to 20 carbon atoms.

Among the (C₆-C₃₀)acyl sarcosinates of formula (I) that may be used in the present composition, mention may be made of palmitoyl sarcosinates, stearoyl sarcosinates, myristoyl sarcosinates, lauroyl sarcosinates and cocoyl sarcosinates, in acid form or in salified form.

The anionic surfactant(s) of sarcosinate type are advantageously chosen from sodium lauroyl sarcosinate, stearoylsarcosine, myristoylsarcosine, and mixtures thereof, preferably from stearoylsarcosine, myristoylsarcosine, and mixtures thereof.

Among the above carboxylic surfactants, mention may also be made of polyoxyalkylenated alkyl(amido) ether carboxylic acids and salts thereof, in particular those including from 2 to 50 alkylene oxide and in particular ethylene oxide groups, such as the compounds sold by the company Kao under the Akypo names.

The polyoxyalkylenated alkyl(amido) ether carboxylic acids that may be used are preferably chosen from those of formula (II):

R₁—(OC₂H₄)ₙ—OCH₂COOA (II)

in which:
R₁ represents a linear or branched C₆-C₂₄ alkyl or alkenyl radical, a (C₈-C₉)alkylphenyl radical, a radical R₂CONH—CH₂—CH₂— with R₂ denoting a linear or branched C₉-C₂₁ alkyl or alkenyl radical;

preferably, R₁ is a C₈-C₂₀ and preferably C₈-C₁₈ alkyl radical, and aryl preferably denotes phenyl,
n is an integer or decimal number (mean value) ranging from 2 to 24 and preferably from 2 to 10,
A denotes H, ammonium, Na, K, Li, Mg or a monoethanolamine or triethanolamine residue.

Use may also be made of mixtures of compounds of formula (II), in particular mixtures of compounds bearing different groups R₁.

The polyoxyalkylenated alkyl(amido) ether carboxylic acids that are particularly preferred are those of formula (II) in which:
R₁ denotes a C₁₂-C₁₄ alkyl, cocoyl, oleyl, nonylphenyl or octylphenyl radical,
A denotes a hydrogen or sodium atom, and
n ranges from 2 to 20, preferably from 2 to 10.

Even more preferentially, use is made of the compounds of formula (II) in which R₁ denotes a C₁₂ alkyl radical, A denotes a hydrogen or sodium atom and n ranges from 2 to 10.

The sulfonate anionic surfactants that may be used include at least one sulfonate function (—SO₃H or —SO₃⁻).

They may be chosen from the following compounds: alkylsulfonates, alkylamidesulfonates, alkylarylsulfonates, α-olefin sulfonates, paraffin sulfonates, alkyl sulfosuccinates, alkyl ether sulfosuccinates, alkylamidesulfosuccinates, alkylsulfoacetates, N-acyltaurates, acylisethionates; alkylsulfolaurates; and also the salts of these compounds;

the alkyl groups of these compounds including from 6 to 30 carbon atoms, notably from 12 to 28, even better still from 14 to 24 or even from 16 to 22 carbon atoms; the aryl group preferably denoting a phenyl or benzyl group;

these compounds possibly being polyoxyalkylenated, notably polyoxyethylenated, and then preferably including from 1 to 50 ethylene oxide units and better still from 2 to 10 ethylene oxide units.

Preferentially, the sulfonate anionic surfactants are chosen, alone or as a mixture, from:
C₆-C₂₄ and notably C₁₂-C₂₀ alkyl sulfosuccinates, notably lauryl sulfosuccinates;
C₆-C₂₄ and notably C₁₂-C₂₀ alkyl ether sulfosuccinates;
(C₆-C₂₄)acylisethionates, preferably (C₁₂-C₁₈)acylisethionates;

in particular in the form of alkali metal or alkaline-earth metal, ammonium or amino alcohol salts.

The sulfate anionic surfactants that may be used include at least one sulfate function (—OSO₃H or —OSO₃⁻).

They may be chosen from the following compounds: alkyl sulfates, alkyl ether sulfates, alkylamido ether sulfates, alkylaryl polyether sulfates, monoglyceride sulfates; and the salts of these compounds;

the alkyl groups of these compounds including from 6 to 30 carbon atoms, notably from 12 to 28, even better still from 14 to 24 or even from 16 to 22 carbon atoms; the aryl group preferably denoting a phenyl or benzyl group;

these compounds possibly being (poly)oxyalkylenated, notably (poly)oxyethylenated, and then preferably including from 1 to 50 ethylene oxide units and better still from 1 to 10 ethylene oxide units.

Preferentially, the sulfate anionic surfactants are chosen, alone or as a mixture, from:
alkyl sulfates, notably C₆-C₂₄ or even C₁₂-C₂₀ alkyl sulfates;
alkyl ether sulfates, notably C₆-C₂₄ or even C₁₂-C₂₀ alkyl ether sulfates, preferably comprising from 1 to 20 ethylene oxide units;

in particular in the form of alkali metal or alkaline-earth metal, ammonium or amino alcohol salts.

When the anionic surfactant is in salt form, said salt may be chosen from alkali metal salts, such as the sodium or potassium salt, ammonium salts, amine salts and in particular amino alcohol salts, and alkaline-earth metal salts, such as the magnesium salt.

Examples of amino alcohol salts that may be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts and in particular the sodium or magnesium salts are preferably used.

Preferentially, the anionic surfactant(s) are chosen from:
C₆-C₃₀ and notably C₈-C₂₄ fatty acids;
C₆-C₂₄ and notably C₁₂-C₂₀ alkyl sulfates;
C₆-C₂₄ and notably C₁₂-C₂₀ alkyl ether sulfates; preferably comprising from 1 to 20 ethylene oxide units;
C₆-C₂₄ alkyl sulfosuccinates and notably C₁₂-C₂₀ alkyl sulfosuccinates; notably lauryl sulfosuccinates;
C₆-C₂₄ and notably C₁₂-C₂₀ alkyl ether sulfosuccinates;
(C₆-C₂₄)acylisethionates, preferably (C₁₂-C₁₈)acylisethionates;
C₆-C₂₄ and notably C₁₂-C₂₀ acylsarcosinates; notably palmitoylsarcosinates, stearoylsarcosinates and myristoylsarcosinates;
(C₆-C₂₄)alkyl ether carboxylates, preferably (C₁₂-C₂₀)alkyl ether carboxylates;
polyoxyalkylenated (C₆-C₂₄)alkyl(amido) ether carboxylic acids and salts thereof, in particular those including from 2 to 50 alkylene oxide and in particular ethylene oxide groups;
C₆-C₂₄ and notably C₁₂-C₂₀ acylglutamates;
C₆-C₂₄ and notably C₁₂-C₂₀ acylglycinates;
and also salts thereof, in particular the alkali metal or alkaline-earth metal or zinc, ammonium or amino alcohol salts thereof;
and mixtures thereof.

Advantageously, the anionic surfactant(s) are chosen from carboxylate anionic surfactants, and mixtures thereof.

The anionic surfactant(s) are preferably chosen from C₆-C₃₀ fatty acids, (C₆-C₃₀)acylglycinates, (C₆-C₃₀)acyllactylates, (C₆-C₃₀)acylsarcosinates, (C₆-C₃₀)acylglutamates; alkyl-D-galactosideuronic acids, alkyl ether carboxylic acids, alkyl(C₆-C₃₀ aryl) ether carboxylic acids, alkylamido ether carboxylic acids; monoesters of C₆-C₂₄ alkyl and of polyglycoside-polycarboxylic acids; and also the salts of these compounds; and mixtures thereof.

The anionic surfactant(s) are advantageously chosen from surfactants based on sulfate or sulfonate functions, and in particular alkyl sulfates, alkyl ether sulfates, alkylamido ether sulfates, alkyl sulfosuccinates, alkyl ether sulfosuccinates and acyl isethionates.

Nonionic Surfactants

The nonionic surfactants that may be used are described, for example, in the Handbook of Surfactants by M. R. Porter, published by Blackie & Son (Glasgow and London), 1991, pages 116-178.

Examples of nonionic surfactants that may be mentioned include the following nonionic surfactants:
oxyalkylenated (C₈-C₂₄)alkylphenols;
saturated or unsaturated, linear or branched, oxyalkylenated or glycerolated C₈ to C₄₀ alcohols, including one or two fatty chains;
saturated or unsaturated, linear or branched, oxyalkylenated C₈ to C₃₀ fatty acid amides;
esters of saturated or unsaturated, linear or branched, C₈-C₃₀ acids and of polyols or polyethylene glycols;
preferably oxyethylenated esters of saturated or unsaturated, linear or branched, C₈ to C₃₀ acids and of sorbitol;
esters of fatty acids and of sucrose;
(C₈-C₃₀)alkyl(poly)glucosides, (C₈-C₃₀)alkenyl(poly)glucosides, which are optionally oxyalkylenated (0 to 10 oxyalkylene units) and comprising from 1 to 15 glucose units, (C₈-C₃₀)alkyl(poly)glucoside esters;
saturated or unsaturated oxyethylenated plant oils;
condensates of ethylene oxide and/or of propylene oxide;
N—(C₈-C₃₀)alkylglucamine and N—(C₈-C₃₀)acylmethylglucamine derivatives;
aldobionamides;
amine oxides;
oxyethylenated and/or oxypropylenated silicones;
and mixtures thereof.

The oxyalkylene units are more particularly oxyethylene or oxypropylene units, or a combination thereof, preferably oxyethylene units.

The number of moles of ethylene oxide and/or of propylene oxide preferably ranges from 1 to 250, more particularly from 2 to 100 and better still from 2 to 50; the number of moles of glycerol notably ranges from 1 to 50 and better still from 1 to 10.

Advantageously, the nonionic surfactants according to the invention do not comprise any oxypropylene units.

As examples of glycerolated nonionic surfactants, use is preferably made of monoglycerolated or polyglycerolated C₈ to C₄₀ alcohols, comprising from 1 to 50 mol of glycerol and preferably from 1 to 10 mol of glycerol.

Among the glycerolated alcohols, it is more particularly preferred to use the C₈/C₁₀ alcohol containing 1 mol of glycerol, the C₁₀/C₁₂ alcohol containing 1 mol of glycerol and the Cu alcohol containing 1.5 mol of glycerol.

The nonionic surfactant(s) that may be used in the composition according to the invention are preferentially chosen from:
oxyethylenated C₈ to C₄₀ alcohols comprising from 1 to 100 mol of ethylene oxide, preferably from 2 to 50 and more particularly from 2 to 40 mol of ethylene oxide and including one or two fatty chains;
saturated or unsaturated oxyethylenated plant oils comprising from 1 to 100 and preferably from 2 to 50 mol of ethylene oxide;
(C₈-C₃₀)alkyl(poly)glucosides, which are optionally oxyalkylenated (0 to 10 EO) and comprising 1 to 15 glucose units;
monoglycerolated or polyglycerolated C₈ to C₄₀ alcohols, comprising from 1 to 50 mol of glycerol and preferably from 1 to 10 mol of glycerol;
saturated or unsaturated, linear or branched, oxyalkylenated C₈ to C₃₀ fatty acid amides;
esters of saturated or unsaturated, linear or branched, C₈-C₃₀ acids and of polyols or polyethylene glycols;
and mixtures thereof.

The nonionic surfactant(s) that may be used are preferably chosen from alkylpolyglucosides.

Amphoteric or Zwitterionic Surfactants

The amphoteric surfactants that may be used in the invention may be optionally quaternized secondary or tertiary aliphatic amine derivatives, in which the aliphatic group is a linear or branched chain including from 8 to 22 carbon atoms, said amine derivatives containing at least one anionic group, for instance a carboxylate, sulfonate, sulfate, phosphate or phosphonate group.

Mention may in particular be made of (C₈-C₂₀)alkylbetaines, sulfobetaines, (C₈-C₂₀)alkylsulfobetaines, (C₈-C₂₀)alkylamido(C₁-C₆)alkylbetaines, such as cocoamidopropylbetaine, (C₈-C₂₀)alkylamido(C₁-C₆)alkylsulfobetaines, and also mixtures thereof.

Among the derivatives of optionally quaternized secondary or tertiary aliphatic amines that may be used, mention may also be made of the products having the respective structures (A1) and (A2) below:

Rₐ—CON(Z)CH₂—(CH₂)ₘ—N⁺(R_{b})(R_{c})(CH₂COO⁻) (A1)

in which:
Rₐ represents a C₁₀-C₃₀ alkyl or alkenyl group derived from an acid Rₐ—COOH preferably present in hydrolysed coconut kernel oil, or a heptyl, nonyl or undecyl group,
R_{b} represents a β-hydroxyethyl group,
R_{c} represents a carboxymethyl group,
m is equal to 0, 1 or 2, and
Z represents a hydrogen atom or a hydroxyethyl or carboxymethyl group;

R_{a′}—CON(Z)CH₂—(CH₂)_{m′}—N(B)(B′) (A2)

in which:
B represents —CH₂CH₂OX′, with X′ representing —CH₂—COOH, CH₂—COOZ′, —CH₂CH₂—COOH, —CH₂CH₂—COOZ′, or a hydrogen atom,
B′ represents —(CH₂)_{z}—Y′, with z=1 or 2, and Y′ representing —COOH, —COOZ′, —CH₂—CHOH—SO₃H or CH₂—CHOH—SO₃Z′,
m′ is equal to 0, 1 or 2,
Z represents a hydrogen atom or a hydroxyethyl or carboxymethyl group,
Z′ represents an ion derived from an alkali metal or alkaline-earth metal, such as sodium, potassium or magnesium; an ammonium ion; or an ion derived from an organic amine and notably from an amino alcohol, such as monoethanolamine, diethanolamine and triethanolamine, monoisopropanolamine, diisopropanolamine or triisopropanolamine, 2-amino-2-methyl-1-propanol, 2-amino-2-methyl-1,3-propanediol and tris(hydroxymethyl)aminomethane, and
R_{a′} represents a C₁₀-C₃₀ alkyl or alkenyl group of an acid R_{a′}COOH preferably present in hydrolysed linseed oil or coconut kernel oil, an alkyl group, notably a C₁₇ alkyl group, and its iso form, or an unsaturated C₁₇ group.

The compounds corresponding to formula (A2) are particularly preferred.

Among the compounds of formula (A2) for which X′ represents a hydrogen atom, mention may be made of the compounds known under the (CTFA) names sodium cocoamphoacetate, sodium lauroamphoacetate, sodium caproamphoacetate and sodium capryloamphoacetate.

Other compounds of formula (A2) are known under the (CTFA) names disodium cocoamphodiacetate, disodium lauroamphodiacetate, disodium caproamphodiacetate, disodium capryloamphodiacetate, disodium cocoamphodipropionate, disodium lauroamphodipropionate, disodium caproamphodipropionate, disodium capryloamphodipropionate, lauroamphodipropionic acid and cocoamphodipropionic acid.

As examples of compounds of formula (A2), mention may be made of the cocoamphodiacetate sold by the company Rhodia under the trade name Miranol® C₂M Concentrate, the sodium cocoamphoacetate sold under the trade name Miranol Ultra C 32 and the product sold by the company Chimex under the trade name Chimexane HA.

Use may also be made of the compounds of formula (A3):

R_{a″}—NH—CH(Y″)—(CH₂)ₙ—C(O)—NH—(CH₂)_{n′}—N(R_{d})(Rₑ) (A3)

in which:
R_{a″} represents a C₁₀-C₃₀ alkyl or alkenyl group of an acid R_{a″}—C(O)OH, which is preferably present in hydrolysed linseed oil or coconut kernel oil;
Y″ represents the group —C(O)OH, —C(O)OZ″, —CH₂—CH(OH)—SO₃H or the group CH₂—CH(OH)—SO₃—Z″, with Z″ representing a cation resulting from an alkali metal or alkaline-earth metal, such as sodium, an ammonium ion or an ion resulting from an organic amine;
R_{d} and Rₑ, independently of each other, represent a C₁-C₄ alkyl or hydroxyalkyl radical; and
n and n′, independently of each other, denote an integer ranging from 1 to 3.

Among the compounds of formula (A3), mention may be made notably of the compound classified in the CTFA dictionary under the name sodium diethylaminopropyl cocoaspartamide and notably the compound sold by the company Chimex under the name Chimexane HB.

The amphoteric surfactant(s) are advantageously chosen from (C₈-C₂₀)alkylbetaines and (C₈-C₂₀)alkylamido(C₁-C₆)alkylbetaines.

When they are present, the total content of the surfactant(s) chosen from cationic, anionic, nonionic and amphoteric or zwitterionic surfactant(s), and mixtures thereof, ranges from 1% to 20% by weight and preferably from 2% to 10% by weight, relative to the total weight of composition (A).

Composition (A) may also comprise one or more liquefied gases.

Preferably, the liquefied gases are chosen from dimethyl ether, chlorinated and/or fluorinated hydrocarbons, such as trichlorofluoromethane, dichlorodifluoromethane, chlorodifluoromethane, 1,1,1,2-tetrafluoroethane, chloropentafluoroethane, 1-chloro-1,1-difluoroethane, 1,1-difluoroethane, tetrafluoropropene; volatile hydrocarbons, which are optionally halogenated, notably such as C₂ to C₅ alkanes, for instance propane, isopropane, n-butane, isobutane, pentane, and mixtures thereof, preferably from C₂ to C₅ alkanes, and mixtures thereof, and more preferentially from propane, isopropane, n-butane, isobutane, and mixtures thereof.

When they are present, the total content of the liquefied gas(es) ranges from 0.5% to 15% by weight, preferably from 1% to 10% by weight, more preferentially from 1.5% to 6% by weight, relative to the total weight of composition (A).

Composition (A) may also comprise one or more fixing polymers, such as the fixing polymers defined below. In the rest of the invention, these fixing polymers of composition (A) are referred to as “additional polymers associated with the rare-earth metals”.

Composition (B)

The process according to the present invention also comprises the application to keratin fibres, in particular to human keratin fibres such as the hair, of a composition (B) comprising one or more fixing polymers.

The fixing polymers or “film-forming polymers” allow the formation of a coating film on the hair, thus providing hairstyle hold.

For the purposes of the invention, the term “fixing polymer” means any polymer that is capable, by application to the hair, of giving a shape to a head of hair or of holding the hair in an already acquired shape.

The fixing polymer(s) that may be used in composition (A) are chosen from anionic fixing polymers, cationic fixing polymers, nonionic fixing polymers and amphoteric fixing polymers, and mixtures thereof.

In the sequential embodiment (or two-step embodiment), composition (B) comprises one or more fixing polymers chosen from anionic fixing polymers.

The anionic fixing polymers generally used are polymers including groups derived from carboxylic, sulfonic or phosphoric acid, and have a number-average molecular mass of between about 500 and 5 000 000.

The carboxylic groups are provided by unsaturated mono- or dicarboxylic acid monomers, such as those corresponding to formula (I):

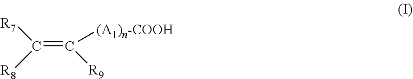

in which n is an integer from 0 to 10, A₁ denotes a methylene group, optionally connected to the carbon atom of the unsaturated group or to the adjacent methylene group when n is greater than 1, via a heteroatom, such as oxygen or sulfur, R₇ denotes a hydrogen atom or a phenyl or benzyl group, R₈ denotes a hydrogen atom or a lower alkyl or carboxyl group, and R₉ denotes a hydrogen atom, a lower alkyl group or a —CH₂—COOH, phenyl or benzyl group.

In the abovementioned formula, a lower alkyl group preferably denotes a group containing 1 to 4 carbon atoms and in particular methyl and ethyl groups.

The anionic fixing polymers containing carboxylic groups that are preferred according to the invention are:

A) copolymers of acrylic or methacrylic acid or salts thereof.

Among these polymers, mention may be made of copolymers of acrylic or methacrylic acid with a monoethylenic monomer, such as ethylene, styrene, vinyl esters or acrylic or methacrylic acid esters, optionally grafted to a polyalkylene glycol, such as polyethylene glycol, and optionally crosslinked. Such polymers are described in particular in French patent 1 222 944 and German patent application 2 330 956, the copolymers of this type including an optionally N-alkylated and/or hydroxyalkylated acrylamide unit in their chain as described notably in Luxembourg patent applications 75370 and 75371. Mention may also be made of copolymers of acrylic acid and of C₁-C₄ alkyl methacrylate and terpolymers of vinylpyrrolidone, of acrylic acid and of C₁-C₂₀ alkyl methacrylate, for example lauryl methacrylate, such as that sold by the company ISP under the name Acrylidone® LM (INCI name: VP/acrylates/lauryl methacrylate copolymer), acrylic acid/ethyl acrylate/N-(t-butyl)acrylamide terpolymers, such as the products Ultrahold® Strong and Ultrahold® 8 sold by the company BASF (INCI name: Acrylates/t-butylacrylamide copolymer), methacrylic acid/ethyl acrylate/tert-butyl acrylate terpolymers, such as the products sold under the names Luvimer® 100 P or Luvimer® PRO 55 by the company BASF (INCI name: Acrylates copolymer), copolymers of methacrylic acid and of ethyl acrylate, such as the products sold under the names Luvimer® MAE or Luviflex® Soft by the company BASF (INCI name: Acrylates copolymer), acrylic acid/butyl acrylate/methyl methacrylate terpolymers, such as the product sold under the name Balance® CR by the company AkzoNobel (INCI name: Acrylates copolymer), or the copolymers of methacrylic acid and of methyl methacrylate sold under the name Eudragit® L 100 by the company Rohm Pharma (INCI name: Acrylates copolymer). Mention may also be made of branched block polymers containing (meth)acrylic acid monomers, such as the product sold under the name Fixate® G-100L by the company Lubrizol (INCI name: AMP-acrylates/allyl methacrylate copolymer);

B) Crotonic acid copolymers, such as those including vinyl acetate or propionate units in their chain and optionally other monomers such as allylic esters or methallylic esters, vinyl ether or vinyl ester of a linear or branched saturated carboxylic acid with a long hydrocarbon-based chain, such as those including at least 5 carbon atoms, these polymers possibly being grafted or crosslinked, or alternatively another vinyl, allylic or methallylic ester monomer of an α- or β-cyclic carboxylic acid. Such polymers are described, inter alia, in French patents 1 222 944, 1 580 545, 2 265 782, 2 265 781, 1 564 110 and 2 439 798. Commercial products which fall into this category are the products Resyn® 28-2930 and 28-1310 sold by the company Akzo Nobel (INCI names: VA/crotonates/vinyl decanoate copolymer and VA/crotonates copolymer, respectively). Mention may also be made of the products Luviset® CA 66 sold by the company BASF, Aristoflex® A60 sold by the company Clariant (INCI name: VA/crotonates copolymer) and Mexomere® PW or PAM sold by the company Chimex (INCI name: VA/vinyl butyl benzoate/crotonates copolymer);

C) copolymers of C₄-C₈ monounsaturated carboxylic acids or anhydrides chosen from:
copolymers comprising (i) one or more maleic, fumaric or itaconic acids or anhydrides and (ii) at least one monomer chosen from vinyl esters, vinyl ethers, vinyl halides, phenylvinyl derivatives, acrylic acid and esters thereof, the anhydride functions of these copolymers optionally being monoesterified or monoamidated. Such polymers are described, in particular, in U.S. Pat. Nos. 2,047,398, 2,723,248 and 2,102,113, and GB patent 839 805. Commercial products are notably those sold under the names Gantrez® AN or ES by the company ISP, such as Gantrez® ES 225 (INCI name: Ethyl ester of PVM/MA copolymer) or Gantrez® ES 425L (INCI name: Butyl ester of PVM/MA copolymer);
copolymers comprising (i) one or more maleic, citraconic or itaconic anhydride units and (ii) one or more monomers chosen from allylic or methallylic esters optionally including one or more acrylamide, methacrylamide, α-olefin, acrylic or methacrylic ester, acrylic or methacrylic acid or vinylpyrrolidone groups in their chain,

the anhydride functions of these copolymers optionally being monoesterified or monoamidated.

These polymers are described, for example, in patents FR 2 350 384 and FR 2 357 241;

D) polyacrylamides including carboxylate groups.

The fixing polymers bearing units derived from sulfonic acid may be chosen from:

A′) homopolymers and copolymers including vinylsulfonic, styrenesulfonic, naphthalenesulfonic or acrylamidoalkylsulfonic units.

These polymers may notably be chosen from:
polyvinylsulfonic acid salts with a molecular mass of between 1000 and 100 000 approximately, and also the copolymers with an unsaturated comonomer such as acrylic or methacrylic acids and esters thereof, and also acrylamide or derivatives thereof, vinyl ethers and vinylpyrrolidone;
polystyrenesulfonic acid salts such as the sodium salts that are sold for example under the name Flexan® II by AkzoNobel (INCI name: Sodium polystyrene sulfonate). These compounds are described in patent FR 2198719;
polyacrylamidosulfonic acid salts, such as those mentioned in patent U.S. Pat. No. 4,128,631, and more particularly the polyacrylamidoethylpropanesulfonic acid sold under the name Rheocare® HSP-1180 by Cognis (INCI name: polyacrylamidomethylpropane sulfonic acid);

B′) sulfonic polyesters, these polymers being advantageously obtained by polycondensation of at least one dicarboxylic acid, of at least one diol or of a mixture of diol and of diamine, and of at least one difunctional monomer including a sulfonic function. Among these polymers, mention may be made of:
linear sulfonic polyesters such as those described in patent applications U.S. Pat. Nos. 3,734,874, 3,779,993, 4,119,680, 4,300,580, 4,973,656, 5,660,816, 5,662,893 and 5,674,479. Such polymers are, for example, the products Eastman® AQ38S Polymer, Eastman® AQ55S Polymer and Eastman® AQ48 Ultra Polymer sold by the company Eastman Chemical (name: Polyester-5) which are copolymers obtained from diethylene glycol, from 1,4-cyclohexanedimethanol, from isophthalic acid and from sulfoisophthalic acid salt;
branched sulfonic polyesters such as those described in patent applications WO 95/18191, WO 97/08261 and WO 97/20899. Such compounds are, for example, the products Eastman® AQ10D Polymer (name: Polyester-13) or Eastman® AQ1350 Polymer sold by the company Eastman Chemical (name: Polyester-13).

According to the invention, the anionic fixing polymer(s) are preferably chosen from acrylic acid copolymers, such as the acrylic acid/ethyl acrylate/N-tert-butylacrylamide terpolymers notably sold under the name Ultrahold® Strong by the company BASF, copolymers derived from crotonic acid, such as the vinyl acetate/vinyl tert-butylbenzoate/crotonic acid terpolymers and the crotonic acid/vinyl acetate/vinyl neododecanoate terpolymers notably sold under the name Resyn 28-2930 by the company AkzoNobel, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, under the names Gantrez® ES 425L or ES 225 by the company ISP, the copolymers of methacrylic acid and of ethyl acrylate sold under the name Luvimer® MAE by the company BASF, and the vinyl acetate/crotonic acid copolymers sold under the name Luviset® CA 66 by the company BASF, and the vinyl acetate/crotonic acid copolymers grafted with polyethylene glycol sold under the name Aristoflex® A60 by the company Clariant, the vinylpyrrolidone/acrylic acid/lauryl methacrylate terpolymers sold under the name Acrylidone® LM by the company ISP, the polymer sold under the name Fixate® G-100L by the company Lubrizol, the vinyl acetate/crotonic acid/vinyl p-tert-butylbenzoate copolymers sold under the names Mexomere® PW or PAM by the company Chimex.

In the sequential embodiment, the fixing polymer(s) are chosen from anionic fixing polymers, more preferentially from fixing polymers bearing carboxylic groups and more particularly based on acrylic or methacrylic monomer and fixing polymers bearing sulfonic groups, and mixtures thereof.

The anionic fixing polymers may be used in a composition that may or may not be predominantly aqueous.

They may be from 0 to 100% neutralized.

The anionic fixing polymer(s) may be in the form of latices or pseudo-latices. In this case, use is made of ingredients such as surfactants, enabling the polymers to be in dispersion form (latex or pseudo-latex).

According to the invention, the term “latex” or “pseudo-latex” means a group of water-insoluble polymers which, when in the form of an aqueous dispersion, are capable of merging to form a film or a coating.

Pseudo-latices are prepared by emulsifying a preformed polymer. For example, pseudolatices are prepared by dissolving the polymer in a suitable solvent and by introducing the organic phase into water to form an emulsion, using an emulsifier, such as sodium lauryl sulfate, and a stabilizer, such as cetyl alcohol. After homogenization, the solvent is removed by vacuum distillation, leaving about 30% of polymer dispersion in water.

A latex is prepared by polymerization of a monomer or a mixture of monomers which is usually emulsified in an aqueous medium containing anionic or nonionic surfactants. The process requires the addition of initiators functioning via radical, anionic or cationic polymerization mechanisms. The polymer latex typically has a particle size of less than a micron.

In the sequential embodiment (or two-step embodiment), composition (B) may also comprise one or more fixing polymers chosen from cationic fixing polymers, nonionic fixing polymers, amphoteric fixing polymers, and mixtures thereof, as described below.

In the one-step embodiment (or all-in-one embodiment), preferably, composition (A+B) contains no (0%) or very little (content of less than 1%) anionic fixing polymer.

In this embodiment, composition (B) comprises one or more fixing polymers chosen from cationic fixing polymers, nonionic fixing polymers and amphoteric fixing polymers, and mixtures thereof.

The cationic fixing polymers that may be used according to the present invention are preferably chosen from polymers including primary, secondary, tertiary and/or quaternary amine groups forming part of the polymer chain or directly attached thereto, and having a molecular weight of between 500 and approximately 5 000 000 and preferably between 1000 and 3 000 000.

Among these polymers, mention may be made more particularly of the following cationic polymers:
(1) homopolymers or copolymers derived from acrylic or methacrylic esters or amides and including at least one of the units of the following formulae:

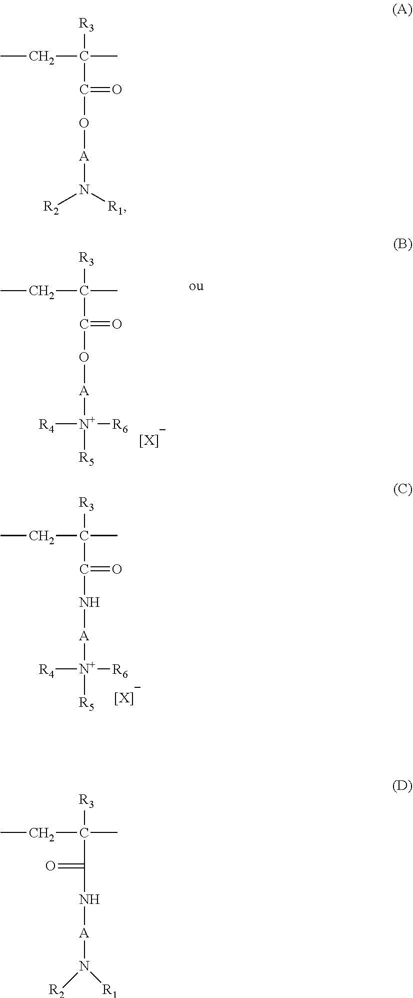

in which:

R₃ denotes a hydrogen atom or a CH₃ radical; A is a linear or branched alkyl group including from 1 to 6 carbon atoms or a hydroxyalkyl group including from 1 to 4 carbon atoms;

R₄, R₅ and R₆, which may be identical or different, represent an alkyl group containing from 1 to 18 carbon atoms or a benzyl radical;

R₁ and R₂, which may be identical or different, each represent a hydrogen atom or an alkyl group containing from 1 to 6 carbon atoms;

X denotes a methosulfate anion or a halide such as chloride or bromide.

The copolymers of class (1) also contain one or more units derived from comonomers which may be chosen from the class of acrylamides, methacrylamides, diacetone acrylamides, acrylamides and methacrylamides substituted on the nitrogen with lower (C₁-C₄) alkyl groups, groups derived from acrylic or methacrylic acids or esters thereof, vinyllactams such as vinylpyrrolidone or vinylcaprolactam, and vinyl esters.

Thus, among these copolymers of class (1), mention may be made of:
quaternized or non-quaternized vinylpyrrolidone/dialkylaminoalkyl acrylate or methacrylate copolymers, such as the products sold under the name Gafquat® by the company ISP, for instance Gafquat® 734 or Gafquat® 755 or Gafquat® 755N (INCI name: Polyquaternium-11), or alternatively the products known as Copolymer® 845, 958 and 937 sold by ISP (INCI name: VP/dimethylaminoethyl methacrylate copolymer). These polymers are described in detail in French patents 2 077 143 and 2 393 573,
polymers bearing a fatty chain and bearing a vinylpyrrolidone unit, such as the products sold under the names Styleze® W20L and Styleze® W10 by the company ISP (INCI name: Polyquaternium-55),
dimethylaminoethyl methacrylate/vinylcaprolactam/vinylpyrrolidone terpolymers, such as the products sold under the names Advantage HC 37 or Gaffix® VC 713 by the company ISP (INCI name: Vinyl caprolactam/VP/dimethylaminoethyl methacrylate copolymer), and
quaternized vinylpyrrolidone/dimethylaminopropylmethacrylamide copolymers, such as the products sold under the name Gafquat® HS 100 by the company ISP (name: Polyquaternium-28);
(2) cationic derivatives of guar gum, preferably comprising quaternary ammonium, such as those described in U.S. Pat. Nos. 3,589,578 and 4,031,307, such as guar gums containing cationic trialkylammonium groups. Such products are notably sold under the trade names Jaguar® C13 S, Jaguar® C 15 and Jaguar® C 17 by the company Rhodia (INCI name: Guar hydroxypropyltrimonium chloride);
(3) quaternary copolymers of vinylpyrrolidone and of vinylimidazole; mention may be made, for example, of vinylpyrrolidone/methyl vinylimidazolium chloride copolymers, such as the products sold by the company BASF under the names Luviquat® FC550 or FC370, Luviquat® Excellence, Luviquat® Style (INCI name: Polyquaternium-16), or vinylpyrrolidone/vinylimidazolium methosulfate/vinylcaprolactam terpolymers, such as the product Luviquat® Hold sold by the company BASF (INCI name: Polyquaternium-46).
(4) chitosans or salts thereof; the salts that may be used are in particular the acetate, lactate, glutamate, gluconate or pyrrolidonecarboxylate of chitosan.

Among these compounds, mention may be made of the chitosan pyrrolidonecarboxylate sold under the name Kytamer® PC by the company Amerchol (INCI name: Chitosan PCA).
(5) cationic cellulose derivatives such as copolymers of cellulose or of cellulose derivatives grafted with a water-soluble monomer including a quaternary ammonium, and described notably in patent U.S. Pat. No. 4,131,576, such as hydroxyalkylcelluloses, for instance hydroxymethyl-, hydroxyethyl- or hydroxypropylcelluloses grafted notably with a methacryloyloxyethyltrimethylammonium, methacrylamidopropyltrimethylammonium or dimethyldiallylammonium salt.

The products sold corresponding to this definition are more particularly the products sold under the names Celquat® L 200 and Celquat® H 100 by the company AkzoNobel (INCI name: Polyquaternium-4).

The amphoteric fixing polymers that may be used in accordance with the invention may be chosen from polymers including units B and C randomly distributed in the polymer chain, where B denotes a unit derived from a monomer including at least one basic nitrogen atom and C denotes a unit derived from an acidic monomer including one or more carboxylic or sulfonic groups or else B and C can denote groups derived from zwitterionic carboxybetaine or sulfobetaine monomers;

B and C may also denote a cationic polymer chain including primary, secondary, tertiary or quaternary amine groups, in which at least one of the amine groups bears a carboxylic or sulfonic group connected via a hydrocarbon-based group or alternatively B and C form part of a chain of a polymer bearing an ethylene-α,β-dicarboxylic unit in which one of the carboxylic groups has been made to react with a polyamine including one or more primary or secondary amine groups.

The amphoteric fixing polymers corresponding to the definition given above that are more particularly preferred are chosen from the following polymers:

(1) copolymers bearing acidic vinyl units and basic vinyl units, such as those resulting from the copolymerization of a monomer derived from a vinyl compound bearing a carboxylic group such as, more particularly, acrylic acid, methacrylic acid, maleic acid, α-chloroacrylic acid, and of a basic monomer derived from a substituted vinyl compound containing at least one basic atom, such as, more particularly, dialkylaminoalkyl methacrylate and acrylate, dialkylaminoalkylmethacrylamide and acrylamide. Such compounds are described in U.S. Pat. No. 3,836,537.

(2) polymers including units derived:
a) from at least one monomer chosen from acrylamides and methacrylamides substituted on the nitrogen atom with an alkyl group,
b) from at least one acidic comonomer containing one or more reactive carboxylic groups, and
c) from at least one basic comonomer such as esters bearing primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides that are more particularly preferred according to the invention are compounds in which the alkyl groups include from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides.

The acidic comonomers are more particularly chosen from acrylic acid, methacrylic acid, crotonic acid, itaconic acid, maleic acid and fumaric acid and alkyl monoesters, containing 1 to 4 carbon atoms, of maleic or fumaric acids or anhydrides.

The preferred basic comonomers are aminoethyl, butylaminoethyl, N,N′-dimethylaminoethyl and N-tert-butylaminoethyl methacrylates.

The copolymers whose INCI name is Octylacrylamide/acrylates/butylaminoethyl methacrylate copolymer, such as the products sold under the names Amphomer®, Amphomer® LV71 or Balance® 47 by the company AkzoNobel, are particularly used.

(3) partially or totally acylated and crosslinked polyaminoamides derived from polyaminoamides of general formula (II):

[symbol]

CO—R₁₀—CO—Z [symbol] (II)

in which R₁₀ represents a divalent group derived from a saturated dicarboxylic acid, from an aliphatic mono- or dicarboxylic acid bearing an ethylenic double bond, from an ester of a lower alkanol containing from 1 to 6 carbon atoms of these acids, or from a group derived from the addition of any one of said acids to a bis-primary or bis-secondary amine, and Z denotes a group derived from a bis-primary, mono- or bis-secondary polyalkylenepolyamine and preferably represents:
a) in proportions of from 60 mol % to 100 mol %, the group (III)

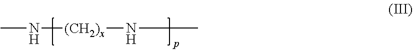

in which x=2 and p=2 or 3, or else x=3 and p=2,

this group being derived from diethylenetriamine, from triethylenetetramine or from dipropylenetriamine;
b) in proportions of from 0 to 40 mol %, the group (III) above in which x=2 and p=1 and which is derived from ethylenediamine, or the group derived from piperazine:

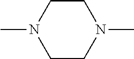

c) in proportions of from 0 to 20 mol %, the —NH—(CH₂)₆—NH— group derived from hexamethylenediamine,

these polyaminoamides being crosslinked by addition reaction of a difunctional crosslinking agent chosen from epihalohydrins, diepoxides, dianhydrides and bis-unsaturated derivatives, using from 0.025 to 0.35 mol of crosslinking agent per amine group of the polyaminoamide and acylated by the action of acrylic acid, chloroacetic acid or an alkane sultone, or salts thereof.

The saturated carboxylic acids are preferably chosen from acids containing 6 to 10 carbon atoms, such as adipic acid, 2,2,4-trimethyladipic acid, 2,4,4-trimethyladipic acid and terephthalic acid, and acids bearing an ethylenic double bond, for instance acrylic, methacrylic and itaconic acids.

The alkane sultones used in the acylation are preferably propane sultone or butane sultone; the salts of the acylating agents are preferably the sodium or potassium salts.

(4) polymers including zwitterionic units of formula (V):

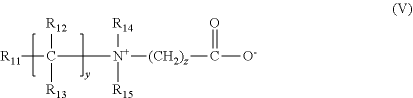

in which R₁₁ denotes a polymerizable unsaturated group, such as an acrylate, methacrylate, acrylamide or methacrylamide group, y and z represent an integer from 1 to 3, R₁₂ and R₁₃ represent a hydrogen atom or a methyl, ethyl or propyl group, and R₁₄ and R₁₅ represent a hydrogen atom or an alkyl group such that the sum of the carbon atoms in R₁₄ and R₁₅ does not exceed 10.

The polymers comprising such units may also include units derived from non-zwitterionic monomers such as dimethyl- or diethylaminoethyl acrylate or methacrylate or alkyl acrylates or methacrylates, acrylamides or methacrylamides or vinyl acetate.

Mention may be made, by way of example, of methyl methacrylate/methyl dimethylcarboxymethylammonioethyl methacrylate copolymers, such as the product sold under the name Diaformer Z-301N or Z-301W by the company Clariant (INCI name: Acrylates copolymer).

(5) polymers derived from chitosan including monomer units corresponding to formulae (E), (F) and (G) below:

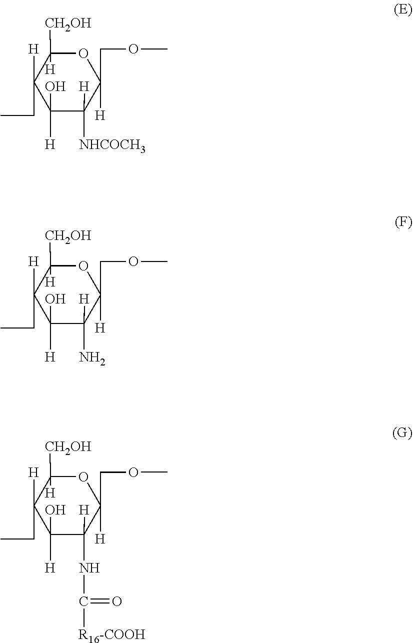

the unit (E) being present in proportions of between 0 and 30%, the unit (F) in proportions of between 5% and 50% and the unit (G) in proportions of between 30% and 90%, it being understood that, in this unit (G), Rib represents a group of formula (VI):

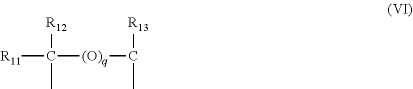

in which
if q=0, R₁₁, R₁₂ and R₁₃, which may be identical or different, each represent a hydrogen atom, a methyl, hydroxyl, acetoxy or amino residue, a monoalkylamine residue or a dialkylamine residue that are optionally interrupted with one or more nitrogen atoms and/or optionally substituted with one or more amine, hydroxyl, carboxyl, alkylthio or sulfonic groups, an alkylthio residue in which the alkyl group bears an amino residue, at least one of the groups R₁₁, R₁₂ and R₁₃ being, in this case, a hydrogen atom; or, if q=1, R₁₁, R₁₂ and R₁₃ each represent a hydrogen atom, and also the salts formed by these compounds with bases or acids.

(6) polymers containing units corresponding to the general formula (VII) are described, for example, in French patent 1 400 366:

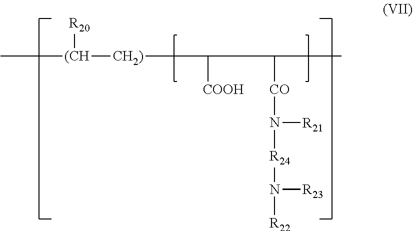

in which R₂₀ represents a hydrogen atom, a CH₃O—, CH₃CH₂O— or phenyl group, R₂₁ denotes a hydrogen atom or a lower alkyl group such as methyl or ethyl, R₂₂ denotes a hydrogen atom or a C₁-C₆ lower alkyl group such as methyl or ethyl, R₂₃ denotes a C₁-C₆ lower alkyl group such as methyl or ethyl or a group corresponding to the formula: —R₂₄—N(R₂₂)₂, with R₂₄ representing a —CH₂—CH₂—, —CH₂—CH₂—CH₂—, or —CH₂—CH(CH₃)— group and R₂₂ having the meanings given above.

(7) polymers derived from the N-carboxyalkylation of chitosan, such as N-carboxymethyl chitosan or N-carboxybutyl chitosan, for instance the product sold under the name Chitoglycan by the company Sinerga SPA (INCI name: Carboxymethyl chitosan).

(8) amphoteric polymers of the -D-X-D-X type chosen from:
a) polymers obtained by the action of chloroacetic acid or sodium chloroacetate on compounds including at least one unit of formula (VIII):

-D-X-D-X-D- (VIII)

in which D denotes a group

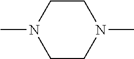

and X denotes the symbol E or E′, where E and E′, which may be identical or different, denote a divalent group that is an alkylene group with a straight or branched chain including up to 7 carbon atoms in the main chain, which is unsubstituted or substituted with hydroxyl groups and which may include, in addition to oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups;
b) polymers of formula (IX):

-D-X-D-X— (IX)

in which D denotes a group

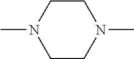

and X denotes the symbol E or E′ and at least once E′; E having the meaning given above and E′ being a divalent group that is an alkylene group with a straight or branched chain containing up to 7 carbon atoms in the main chain, which is unsubstituted or substituted with one or more hydroxyl groups and which includes one or more nitrogen atoms, the nitrogen atom being substituted with an alkyl chain that is optionally interrupted with an oxygen atom and which necessarily includes one or more carboxyl functions or one or more hydroxyl functions betainized by reaction with chloroacetic acid or sodium chloroacetate;

(9) (C₁-C₅)alkyl vinyl ether/maleic anhydride copolymers partially modified by semiamidation with an N,N-dialkylaminoalkylamine, such as N,N-dimethylaminopropylamine, or by semiesterification with an N,N-dialkylaminoalkanol. These copolymers may also include other vinyl comonomers, such as vinylcaprolactam.

Among the amphoteric fixing polymers mentioned above, the ones that are most particularly preferred according to the invention are those of family (3), such as the copolymers whose INCI name is octylacrylamide/acrylates/butylaminoethyl methacrylate copolymer, such as the products sold under the names Amphomer®, Amphomer® LV 71 or Balance® 47 by the company AkzoNobel and those of family (4) such as the copolymers of methyl methacrylate/methyl dimethylcarboxymethylammonioethyl methacrylate, sold, for example, under the name Diaformer Z-301N or Z-301W by the company Clamant.

The nonionic fixing polymers that may be used according to the present invention are chosen, for example, from:
polyalkyloxazolines;
vinyl acetate homopolymers,
vinyl acetate copolymers, for instance copolymers of vinyl acetate and of acrylic ester, copolymers of vinyl acetate and of ethylene, or copolymers of vinyl acetate and of maleic ester, for example of dibutyl maleate;
homopolymers and copolymers of acrylic esters, for instance copolymers of alkyl acrylates and of alkyl methacrylates, such as the products sold by the company Röhm GmbH under the name Eudragit® NE 30 D (INCI name: Acrylates copolymer);
copolymers of acrylonitrile and of a nonionic monomer chosen, for example, from butadiene and alkyl (meth)acrylates;
styrene homopolymers;
styrene copolymers, for instance copolymers of styrene, of alkyl acrylate and of alkyl methacrylate; copolymers of styrene and of butadiene; or copolymers of styrene, of butadiene and of vinylpyridine;
polyamides;
vinyllactam homopolymers, such as the vinylpyrrolidone homopolymers sold, for example, under the names Luviskol® K30 Powder by the company BASF or PVP K30L or K60 Solution or K90 by the company ISP, or such as the polyvinylcaprolactam sold under the name Luviskol® Plus by the company BASF (INCI name: PVP);
vinyllactam copolymers, such as a poly(vinylpyrrolidone/vinyllactam) copolymer sold under the trade name Luvitec® VPC 55K65W by the company BASF, poly(vinylpyrrolidone/vinyl acetate) copolymers, such as those sold under the name PVP/VA® S630L, E735, E635 and W735 by the company ISP, Luviskol® VA 73, VA 64 and VA 37 by the company BASF (INCI name: VP/VA copolymer); and vinylpyrrolidone/methacrylamide/vinylimidazole terpolymers, for instance the product sold under the name Luviset® Clear by the company BASF (INCI name: VP/methacrylamide/vinyl imidazole copolymer).

The alkyl groups of the nonionic polymers mentioned above preferably contain from 1 to 6 carbon atoms.

Use may also be made, according to the invention, of fixing polymers of grafted silicone type comprising a polysiloxane portion and a portion composed of a non-silicone organic chain, one of the two portions constituting the main chain of the polymer and the other being grafted onto said main chain.

These polymers are described, for example, in patent applications EP-A-0 412 704, EP-A-0 412 707, EP-A-0 640 105 and WO 95/00578, EP-A-0 582 152 and WO 93/23009, and patents U.S. Pat. Nos. 4,693,935, 4,728,571 and 4,972,037.

These polymers may be amphoteric, anionic or nonionic and they are preferably anionic or nonionic.

Such polymers are, for example, copolymers that may be obtained by free radical polymerization from the monomer mixture formed from:

a) 50% to 90% by weight of tert-butyl acrylate,

b) 0 to 40% by weight of acrylic acid,

c) 5% to 40% by weight of a silicone macromer of formula:

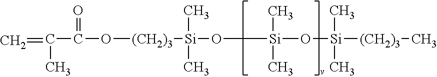

in which v is a number ranging from 5 to 700, the weight percentages being calculated relative to the total weight of the monomers.

Other examples of grafted silicone polymers are notably polydimethylsiloxanes (PDMSs) to which are grafted mixed polymer units of the poly((meth)acrylic acid) type and of the poly(alkyl (meth)acrylate) type via a thiopropylene-type connecting chain and polydimethylsiloxanes (PDMSs) to which polymer units of the poly(isobutyl (meth)acrylate) type are grafted via a thiopropylene-type connecting chain.

Grafted silicone polymers are sold, for example, under the names Silicone Plus Polymer® VS80 and VA70 by 3M (INCI names: Polysilicone-8 and Polysilicone-7, respectively).

Another type of silicone fixing polymer that may be mentioned is the product Luviflex® Silk sold by the company BASF (INCI name: PEG/PPG-25/25 dimethicone/acrylates copolymer).

Functionalized or non-functionalized, silicone or non-silicone, cationic, nonionic, anionic or amphoteric polyurethanes or mixtures thereof may also be used as fixing polymers.

The polyurethanes particularly targeted by the present invention are those described in patent applications EP 0 751 162, EP 0 637 600, EP 0 648 485 and FR 2 743 297, of which the applicant is the proprietor, and also in patent applications EP 0 656 021 and WO 94/03510 from the company BASF and EP 0 619 111 from the company National Starch.

As polyurethanes that are particularly suitable for use in the present invention, mention may be made of the products sold under the names Luviset PUR® and Luviset® Si PUR by the company BASF (INCI names: Polyurethane-1 and Polyurethane-6, respectively).

One or more anionic fixing polymers may also be used in the one-step process. The anionic fixing polymers are then formulated so that they are mildly neutralized (<25%) and preferentially in the form of a mixture of a composition A and of a composition B at the time of use.

The anionic fixing polymers may also be used in a composition containing a minor amount of water (water <50%), or even with little water (water <10%), or even water-free. In this case, the solvent is an organic molecule or a mixture of organic molecules (ethanol, isopropanol, acetone or MEK).

In the sequential process, advantageously, the total content of the fixing polymer(s) ranges from 0.1% to 60%, relative to the total weight of composition (B).

In the sequential process, advantageously, the total content of the anionic fixing polymer(s) ranges from 0.1% to 60%, relative to the total weight of composition (B).

In the one-step process, advantageously, the total content of the fixing polymer(s) ranges from 0.1% to 45%, relative to the total weight of composition (A+B).

In the one-step process without mixing compositions (A) and (B) at the time of use (i.e. compositions (A) and (B) are mixed beforehand), advantageously, the content of the compound(s) of a metal belonging to the group of the rare-earth metals ranges from 0.025% to 15% by weight and the content of the polymer(s) ranges from 0.5% to 15% by weight relative to the total weight of composition (A+B). Preferably, the weight ratio between the content of the compound(s) of a metal belonging to the group of the rare-earth metals and the content of the polymer(s) ranges from 0.2 to 5.

Composition (B) is preferably aqueous, alcoholic or aqueous-alcoholic.

When composition (B) is aqueous, it preferably comprises water, in a content of greater than or equal to 50% by weight, more preferentially greater than or equal to 70% by weight, and even more preferentially greater than or equal to 90% by weight relative to the total weight of composition (B).

When composition (B) is alcoholic or aqueous-alcoholic, it may notably comprise one or more organic solvents, preferably in a content ranging from 0.05% to 95% by weight and even more preferentially from 1% to 70% by weight, relative to the total weight of composition (B).

This organic solvent may be a C₂ to C₄ lower alcohol, in particular ethanol or isopropanol, polyols and polyol ethers such as propylene glycol, polyethylene glycol or glycerol. The organic solvent is preferably ethanol.

Composition (B) may also comprise one or more surfactants chosen from cationic, anionic, nonionic and amphoteric or zwitterionic surfactants, and mixtures thereof, such as the surfactants described previously in composition (A).

In the case of the sequential process, composition (A) has a pH preferably less than 7, more preferentially less than 6. In this embodiment, composition (B) has a pH preferably ranging from 3 to 13.

In the case of the one-step process with mixing of compositions (A) and (B) at the time of use, composition (A) has a pH preferably less than 7, more preferentially less than 6, and composition B is made so that the pH of the mixture (A+B) is preferably less than 7 and more preferentially less than 6.

In the case of the one-step process without mixing of compositions (A) and (B) at the time of use, the composition has a pH preferably less than 7 and more preferentially less than 6.

Additives

Compositions (A) and (B) according to the present invention may also optionally comprise one or more additives, other than the compounds of the invention and among which mention may be made of cationic, anionic, nonionic and amphoteric polymers other than the fixing polymers of the invention, or mixtures thereof, antidandruff agents, anti-seborrhoea agents, vitamins and provitamins including panthenol, sunscreens, sequestrants, plasticizers, solubilizers, acidifying agents, alkaline agents, mineral or organic thickeners, notably polymeric thickeners, antioxidants, hydroxy acids, fragrances and preserving agents.

Needless to say, a person skilled in the art will take care to select this or these optional additional compounds such that the advantageous properties intrinsically associated with the composition according to the invention are not, or are not substantially, adversely affected by the envisaged addition(s).

The above additives may generally be present in an amount, for each of them, of between 0 and 20% by weight relative to the total weight of compositions (A) and/or (B).

Implementation of the Process

The cosmetic process for treating keratin fibres, in particular human keratin fibres such as the hair, according to the present invention comprises:
application to said keratin fibres of a composition (A) comprising one or more compounds of a metal belonging to the group of the rare-earth metals in oxidation state +III as defined above; and
application to said keratin fibres of a composition (B) comprising one or more fixing polymers as defined above,

it being understood that composition (A) and composition (B) are applied to said keratin fibres together or sequentially.

The cosmetic process of the invention is in particular a process for styling and/or shaping said keratin fibres.

In one embodiment, compositions (A) and (B) are applied together, i.e. “one-step process” or all-in-one process”, the compositions possibly being mixed beforehand (i.e. one-step process without mixing at the time of use) or at the time of use.

In another embodiment, compositions (A) and (B) are applied sequentially, i.e. “two-step process” or “sequential process”. In this embodiment, composition (A) may be applied before or after composition (B).

According to a first preferred embodiment, known as the “all-in-one” embodiment, composition (A) and composition (B) as defined previously are applied together (or jointly), i.e. simultaneously, to the keratin fibres or with prior mixing, preferentially performed just before the application, for example by using a double tube. According to this embodiment, the cosmetic treatment process is performed in one step.

Advantageously, according to this embodiment, compositions (A) and (B) as defined previously are mixed and the resulting composition is then applied to the keratin fibres. Preferably, in this embodiment, the fixing polymer(s) are chosen from cationic fixing polymers, nonionic fixing polymers, amphoteric fixing polymers, and mixtures thereof.

In the all-in-one process, composition (A+B) may preferably be left to stand on the keratin fibres for a time ranging from less than 1 to 15 minutes and more preferentially for a time ranging from 1 to 5 minutes.

According to a second preferred embodiment, known as the “sequential process”, composition (A) and composition (B) as defined previously are applied sequentially, i.e. successively. According to this second embodiment, the cosmetic treatment process is performed in at least two steps.

According to a first variant of this second embodiment, composition (A) is applied to the keratin fibres before composition (B).

According to this first variant, the cosmetic process for treating keratin fibres, in particular human keratin fibres such as the hair, according to the present invention comprises at least the following two successive steps:
a first application to said keratin fibres of a composition (A) comprising one or more compounds of a metal belonging to the group of the rare-earth metals in oxidation state +III as defined above; followed by
a second application to said keratin fibres of a composition (B) comprising one or more fixing polymers, preferably chosen from anionic fixing polymers, as defined above.

According to a second variant of this second embodiment, composition (B) is applied to the keratin fibres before composition (A).

According to this second variant, the cosmetic process for treating keratin fibres, in particular human keratin fibres such as the hair, according to the present invention comprises at least the following two successive steps:
a first application to said keratin fibres of a composition (B) comprising one or more fixing polymers, preferably chosen from anionic fixing polymers, as defined above; followed by
a second application to said keratin fibres of a composition (A) comprising one or more compounds of a metal belonging to the group of the rare-earth metals in oxidation state +III as defined above.

In the sequential process, each of the compositions (A) and (B) may preferably be left to stand on the keratin fibres for a time ranging from 1 to 15 minutes and more preferentially for a time ranging from 1 to 5 minutes.

Moreover, the duration between the two steps is preferably less than or equal to 1 hour after step a), more preferentially less than or equal to 30 minutes, even more preferentially less than or equal to 15 minutes.

The process according to the invention may be performed on wet or dry keratin fibres. The keratin fibres may optionally have undergone washing and/or rinsing beforehand.

Advantageously, the process according to the invention is followed by a step of shaping the keratin fibres, and notably the hair.

The invention also relates to a multi-compartment kit or device comprising a first compartment containing a composition (A) as defined previously; and a second compartment containing a composition (B) as defined previously. This kit or device makes it possible to vary the amount of fixing polymer(s) in the product applied (if performed in one step) or the relative amounts of composition (B) relative to composition (A) applied (if performed sequentially). Typically, the device or kit makes it possible to reduce the content of fixing polymers in the course of the applications.

For example, in the case of a sequential process, a process consisting in applying a composition (A) containing from 0.1% to 10% of compounds of a metal belonging to the group of the rare-earth metals in oxidation state +III and then a composition (B) containing more than 5% of fixing polymers is performed to begin with. Next, in the course of the applications, the process evolves towards a process consisting in applying a composition (A) containing from 0.1% to 10% of compounds of a metal belonging to the group of the rare-earth metals in oxidation state +III and application of a composition (B) with less than 5% of polymer.

For example, in the case of an all-in-one process, a process consisting in applying a composition comprising more than 5% of fixing polymers and 0.1% to 10% of compounds of a metal belonging to the group of the rare-earth metals in oxidation state +III is performed to begin with. Next, in the course of the applications, the process evolves towards a process consisting in applying a composition comprising less than 5% of fixing polymers and 0.1% to 10% of compounds of a metal belonging to the group of the rare-earth metals.

To perform these processes, use may advantageously be made of a composition (A) with little or no “additional polymers associated with rare-earth metals”.

The examples that follow serve to illustrate the invention without, however, being limiting in nature.

### EXAMPLES

In the examples that follow, all the amounts are given, unless otherwise indicated, as mass percentages of active material relative to the total weight of the composition.

### Example 1 (Sequential Process)

a) Compositions

Compositions A1, A0 and B1, B2, B3 and B0 were prepared from the ingredients whose contents are indicated in the table below:

TABLE 1

| | Composition A1 | Composition A0 |
|---|---|---|
| Hydrated cerium chloride⁽¹⁾ | 2.5 | — |
| NaCl | — | 2.5 |
| Water | qs 100 | qs 100 |
| pH (citric acid qs) | 4.5 | 4.5 |

⁽¹⁾sold under the name CeCl₃•7H₂O by Sigma-Aldrich

TABLE 2

| | Composition | Composition | Composition | Composition |
|---|---|---|---|---|
| | B1 | B2 | B3 | B0 |
| copolymer of | 5 | — | 5 | — |
| vinyl acetate, | | | | |
| vinyl para-tert- | | | | |
| butylbenzoate | | | | |
| and crotonic acid⁽²⁾ | | | | |
| 100% | | | | |
| neutralized with | | | | |
| AMP | | | | |
| non-neutralized | — | 5 | — | — |
| copolymer of | | | | |
| vinyl acetate, | | | | |
| vinyl para-tert- | | | | |
| butylbenzoate | | | | |
| and crotonic | | | | |
| acid⁽²⁾ | | | | |
| Pure ethanol | — | qs 100 | qs 100 | — |
| Water | qs 100 | — | — | qs 100 |

⁽²⁾sold under the name Mexomer PW by Chimex

b) Procedure and Results

Malleable heads are prepared by cutting the hair in a mid-length cut, i.e. with the hair arriving to the base of the neck.

The following process is performed on the right side of a first head T1:

1 g of composition A1 is applied with a pipette to the roots and the hair is then combed from the root to the ends to make the composition penetrate and spread. This is repeated nine times in a row, taking care to treat the majority of the hair of this side of the malleable head.

Composition B1 is then applied in an amount of 2 g of composition B1. This application is repeated 10 times in total. Next, after a leave-on time of 15 minutes, the head of hair is rinsed thoroughly with 4 litres of water.

The same process as previously is performed on the left side of the same head T1, replacing composition A1 with composition A0.

Before the hair has dried, it is combed, taking care to place the hair horizontally. To do this, each lock is taken in the teeth of the comb at the root and the comb is then slid towards the ends, forcing the hair backwards. The hair is then left to dry naturally.

It is observed that, in the two sides of the malleable head T1, the hair strands are then set in shape (hair back-combed).

The hair is then washed. Just after, the hair that is back-combed is put back in place with the comb, and is then left to dry.

It is observed that the hair remains in the horizontal shape on the right side (treated with the composition containing a rare-earth metal salt), whereas it regains a vertical position on the left side (treated with the composition without the rare-earth metal salt).

The same tests are performed on the heads T2 and T3 for which composition B1 was replaced with compositions B2 and B3, respectively.

Next, four new cycles of application of the respective compositions A and B and of washing are performed on the heads T1, T2 and T3.

It is observed that the styling effect observed after washing on the right side of each head (treated with the composition containing a rare-earth metal salt) becomes accentuated in the course of the cycles.

The following process is performed on the right side of a head T4:

1 g of composition A1 is applied with a pipette to the roots and the hair is then combed from the root to the ends to make the composition penetrate and spread. This is repeated nine times in a row, taking care to treat the majority of the hair of this side of the malleable head.

Composition B1 is then applied in an amount of 2 g of composition B1. This application is repeated 10 times in total. Next, after a leave-on time of 15 minutes, the head of hair is rinsed thoroughly with 4 litres of water.

The same process as previously is performed on the left side of the same head T4, replacing composition B1 with composition B0.

It is observed after drying that the hair maintains its horizontal shape on the right side and that the hair regains a vertical position on the left side.

Heads T1 to T4 are taken again and five washing cycles are performed. It is observed on the four heads, on the right side, that the hair is cleared and is much easier to style than on the left side (in the sense that, once the horizontal style has been given, the shape is particularly resistant).

### Example 2 (All-In-One Process)

a) Compositions

Compositions C1, C2, C3, C4 and C5 were prepared from the ingredients whose contents are indicated in the table below:

TABLE 3

| | C1 | C2 | C3 | C4 | C5 |
|---|---|---|---|---|---|
| Hydrated cerium chloride⁽¹⁾ | 2.5 | 2.5 | 2.5 | 2.5 | 1.5 |
| Hydroxypropylcellulose⁽³⁾ | 2.5 | — | — | — | 1.5 |
| Polyvinyl alcohol⁽⁴⁾ | — | 2.5 | — | — | — |
| Polyvinylpyrrolidone⁽⁵⁾ | — | — | 2.5 | — | — |
| 50% neutralized P4VP | — | — | — | 2.5 | — |
| Water | qs 100 | qs 100 | qs 100 | qs 100 | qs 100 |
| PH | 4.5 | 4.8 | 5.1 | 5.1 | 4.6 |

⁽¹⁾sold under the name CeCl₃•7H₂O by Sigma-Aldrich
⁽³⁾sold under the name Klucel EF Pharm Hydroxypropylcellulose by Ashland
⁽⁴⁾sold under the name Selvol Ultalux FF by Sekisui Speciality
⁽⁵⁾sold under the name PVPK by Ashland

Compositions C1′, C2′, C3′, C4′ and C5′ corresponding to C1, C2, C3, C4 and C5 are prepared, in which no rare-earth metal salt is used, and are adjusted to the same pH using citric acid.

Five malleable heads of bleached (Blondys bleach) mid-length Caucasian hair are prepared.

On the first malleable head:

Day 1: the left side is treated with composition C1 and the right side is treated with composition C1′, in a proportion of 8 g per half-head. The hair is shaped by back-combing as in Example 1 and is then left to dry. At this stage, the styling effect is graded as in Example 1 and the natural appearance of the style is graded. The hair is then washed (with a single shampooing time) and dried, and the quality of the hair is graded: body, cleanliness of the hair and possibility of putting the back-combed hair back in place.

Days 2 to 10: the same procedure is repeated and evaluations are made on Day 5 (D5) and Day 10 (D10).

The grades range from 0 to 5 in increments of 0.5, 0 being the worst grade and 5 the best.

The results of these evaluations are presented in the table below:

TABLE 4

| | | Natural | | | |
|---|---|---|---|---|---|
| | | appearance | Cleanliness of | | Possibility of |
| | Hold of the given | of the hairstyle | the hair (after | Body (after | restyling (with |
| | shape (before | (before | washing and | washing, drying | washing, drying |
| | shampooing) | shampooing) | drying) | and combing) | and combing) |
| D1-left side | 4.1 | 2.1 | 4.6 | 2.2 | 2.1 |
| D1-right side | 4 | 2.1 | 4.5 | 1.9 | 1.5 |
| D5-left side | 4.4 | 2.1 | 4.6 | 3.4 | 3.9 |
| D5-right side | 4 | 2.0 | 4.6 | 1.9 | 1.5 |
| D10-left side | 4.5 | 2.4 | 4.3 | 3.9 | 4.2 |
| D10-right side | 4 | 2.0 | 4.3 | 1.9 | 1.5 |

Better body of the hair and better possibility of restyling is observed on the left side (invention) relative to the right side (comparative) on days 1, 5 and 10. A more substantial styling effect is observed on the left side (invention) on days 5 and 10.

On the other four malleable heads, the same protocol as above is performed, comparing the following compositions, respectively: C2 versus C2′, C3 versus C3′, C4 versus C4′ and C5 versus C5′.

The same effects as for the first malleable head are observed, namely a substantial immediate styling effect (before shampooing) on both sides (left and right) and a substantial styling effect after washing and drying on the left side (invention) but not on the right side (comparative).

Thereafter, the first malleable head is treated according to the same protocol, using composition C6 (below) on the left side, and composition C1′ on the right side.

The cycles of application of the compositions and washing are continued up to day 15.

The same evaluations as previously are performed on days 11 (D11) and 15 (D15).

TABLE 5

| | Composition C6 |
|---|---|
| Hydrated cerium chloride⁽¹⁾ | 2.5 |
| Hydroxypropylcellulose⁽³⁾ | 0.5 |
| Water | qs 100 |
| PH | 4.5 |

⁽¹⁾sold under the name CeCl₃•7H₂O by Sigma-Aldrich
⁽³⁾sold under the name Klucel EF Pharm Hydroxypropylcellulose by Ashland

The results of these evaluations are presented in the table below:

| | | Natural | | | |
|---|---|---|---|---|---|
| | | appearance | | | |
| | | of the head | Cleanliness of | | Possibility of |
| | Hold of the given | of hair | the hair (after | Body (after | restyling (with |
| | shape (before | (before | washing and | washing, drying | washing, drying |
| | shampooing) | shampooing) | drying) | and combing) | and combing) |
| D11-left side | 3.5 | 4 | 4.5 | 3.9 | 4.1 |
| D11-right side | 4 | 2.0 | 4.3 | 1.9 | 1.5 |
| D15-left side | 4 | 4 | 4.6 | 4.0 | 4.0 |
| D15-right side | 4 | 2.0 | 4.5 | 1.9 | 1.5 |

### Example 3

a) Compositions

The preceding composition C1 and composition C7 are prepared from the ingredients whose contents are indicated in the table below:

TABLE 7

| | Composition C7 |
|---|---|
| Hydrated cerium chloride⁽¹⁾ | 2.5 |
| Water | qs 100 |
| PH | 4.7 |

⁽¹⁾sold under the name CeCl₃•7H₂O by Sigma-Aldrich

b) Procedure and Results

Compositions C1 and C7 may be mixed in various relative proportions.

In a first stage, the hair is treated with composition C1. After a few days, the hair is treated with a mixture of 50% of composition C1 and 50% of composition C₇. After a few more days, the hair is once again treated with composition C1.

It is possible to adapt the relative proportions of compositions C1 and C7 in order to obtain the mixture that is the most efficient as a function of the head of hair.

### Example 4

a) Compositions

Compositions A3, A4 and A5 and B4 were prepared from the ingredients whose contents are indicated in the table below:

TABLE 8

| | A3 | A4 | A5 | B4 |
|---|---|---|---|---|
| Hydrated yttrium | 5 | — | — | — |
| chloride⁽⁶⁾ | | | | |
| Hydrated lanthanum | — | 5 | — | — |
| chloride⁽⁷⁾ | | | | |
| Hydrated calcium | — | — | 5 | — |
| chloride⁽⁸⁾ | | | | |
| Hydroxypropylcellulose⁽³⁾ | — | — | — | 2 |
| Water | qs 100 | qs 100 | qs 100 | qs 100 |
| pH (citric acid qs) | 4.5 | 4.5 | 4.4 | 4.5 |

(6)sold under the name YCl₃•6H₂O by Sigma-Aldrich
⁽⁷⁾sold under the name LaCl₃•7H₂O by Sigma-Aldrich
⁽⁸⁾CaCl₂•2H₂O
⁽³⁾sold under the name Klucel EF Pharm Hydroxypropylcellulose by Ashland

Compositions M1, M2, M3 and M4 were then prepared at the time of use by mixing, respectively, 50% of the compositions A1, A3, A4 and A5 with 50% of composition B4.

b) Procedure and Results

Three malleable heads are prepared as in Example 1.

The following process is performed on the right side of a first malleable head (same preparation as in Example 1):

1 g of composition M1 is applied with a pipette to the roots and the hair is then combed from the root to the ends to make the composition penetrate and spread.

This is repeated nine times in a row, taking care to treat the majority of the hair of this side of the malleable head.

The same is done on the left side with composition M2.

Composition M1 is applied on the right and composition M3 is applied on the left, as previously, on the second malleable head.

Composition M1 is applied on the right and composition M4 is applied on the left, as previously, on the third malleable head.

The hold of the shape once the hair is dry is noted each time.

For the first two malleable heads, the hold is better on the left, the rare-earth metals yttrium and lanthanum afford better results in terms of hold of the shape of the hair than the rare-earth metal cerium.

For the third malleable head, better hold is observed on the right, the rare-earth metal cerium thus affords a better result than calcium.

1. Cosmetic process for treating keratin fibres, in particular human keratin fibres such as the hair, comprising:
application to said keratin fibres of a composition (A) comprising one or more salts and/or complexes of a metal belonging to the group of the rare-earth metals in oxidation state +III; and
application to said keratin fibres of a composition (B) comprising one or more fixing polymers,
it being understood that composition (A) and composition (B) are applied to said keratin fibres together or sequentially.

2. Process according to claim 1, characterized in that the metal belonging to the group of the rare-earth metals is chosen from cerium, yttrium, ytterbium, lanthanum and europium.

3. Process according to claim 1, characterized in that the salts and/or complexes of a metal belonging to the group of the rare-earth metals in oxidation state +III are chosen from cerium chloride, yttrium chloride, lanthanum chloride, ytterbium chloride, europium chloride, and mixtures thereof.

4. Process according to claim 1, characterized in that the metal compound(s) are present in a content ranging from 0.05% to 25% by weight and preferably from 0.2% to 15% by weight relative to the total weight of composition (A).

5. Process according to claim 1, characterized in that the fixing polymer(s) are chosen from anionic fixing polymers, nonionic fixing polymers, cationic fixing polymers and amphoteric fixing polymers, and mixtures thereof.

6. Process according to claim 1, characterized in that the fixing polymer(s) are present in a total content ranging from 0.1% to 60% by weight relative to the total weight of composition (B).

7. Process according to claim 1, characterized in that the fixing polymer(s) are chosen from anionic fixing polymers, more preferentially from fixing polymers bearing carboxylic groups and more particularly based on acrylic monomer and fixing polymers bearing sulfonic groups, and mixtures thereof.

8. Process according to claim 1, characterized in that composition (A) and composition (B) are applied sequentially to the keratin fibres.

9. Process according to claim 1, characterized in that composition (A) is applied before composition (B).

10. Process for treating keratin fibres according to claim 1, characterized in that composition (B) is applied before composition (A).

11. Process according to claim 1, characterized in that the fixing polymer(s) are chosen from nonionic fixing polymers, cationic fixing polymers and amphoteric fixing polymers, and mixtures thereof.

12. Process according to claim 1, characterized in that composition (A) and composition (B) are applied together to the keratin fibres.

13. Multi-compartment kit or device comprising a first compartment containing a composition (A) and a second compartment containing a composition (B).
